(19) United States
(12) Patent Application Publication
(10) Pub. No.: US 2013/0210861 A1
(43) Pub. Date: Aug. 15, 2013

(54) METHODS AND COMPOSITIONS FOR MANAGING CARDIOVASCULAR DISEASE ASSOCIATED WITH CHRONIC KIDNEY DISEASE

(71) Applicants: Ashutosh M. SHUKLA, Little Rock, AR (US); Sudhir V. SHAH, Little Rock, AR (US)

(72) Inventors: Ashutosh M. SHUKLA, Little Rock, AR (US); Sudhir V. SHAH, Little Rock, AR (US)

(21) Appl. No.: 13/709,057

(22) Filed: Dec. 9, 2012

Related U.S. Application Data
(60) Provisional application No. 61/569,173, filed on Dec. 9, 2011.

(51) Int. Cl. A61K 31/4706

(52) U.S. Cl. CPC A61K 31/4706
USPC 514/313

(57) ABSTRACT

Methods of treating cardiovascular disease in patients predisposed to or afflicted with kidney disease, particularly chronic kidney disease, are described. In such methods, therapeutically effective amounts of chloroquine-based compounds, for example, hydroxychloroquine, are administered to prevent or otherwise ameliorate arthrosclerosis and other cardiovascular diseases.

### RELATED APPLICATION

This application claims the benefit of and priority to commonly owned U.S. provisional patent application Ser. No. 61/569,173, filed 9 Dec. 2011, which is hereby incorporated by reference in its entirety for any and all purposes.

### STATEMENT REGARDING FEDERALLY SPONSORED RESEARCH

Work relating to this application was supported by a grant from the U.S. Government (VA MERIT Review ACUC#8-07-4). The government may have certain rights in the invention.

### FIELD OF THE INVENTION

This invention relates to management of cardiovascular disease. More particularly, this invention relates to treating patients in need thereof, such as patients with chronic kidney disease, with compositions comprising chloroquine and related compounds to ameliorate and/or lessen the occurrence of cardiovascular disease in such patients.

### BACKGROUND OF THE INVENTION

The following description in this Background section includes information that may be useful in understanding the present invention. It is not an admission that any such information is prior art, or relevant, to the presently claimed inventions, or that any publication specifically or implicitly referenced is prior art.

Cardiovascular Disease (CVD) is the cause of the highest annual mortality for U.S. population over the last century and is associated with very high health care costs. Management of traditional risk factors in diseases such as CVD has been a long-term goal with current treatment methodologies providing only partial benefit in CVD related mortality.

Chronic Kidney Disease (CKD) is a major non-traditional risk factor for cardiovascular disease exhibiting accelerated atherosclerosis (AS) and exponentially rising cardiovascular mortality. Moderate to severe CKD is prevalent in nearly 8% of the US population having increased at a three-fold rate over the past ten years as measured by USRDS (2009). CKD of at least stage III severity affects approximately 9-13% of US population by various estimates. CKD accounts for close to 28% of overall Medicare spending. With regard to patients with CKD that experience Cardiovascular Disease, Medicare costs have already increased to $54 billion dollars as of 2007 and that level of spending represents nearly 32% of Medicare CVD costs. Further, the rising epidemic of obesity, diabetes and metabolic syndrome is expected to result in increased presentation of CKD in patients so afflicted and a concomitant increase in costs to health care management.

CVD is highly prevalent in patients with any degree of CKD and its mortality rises exponentially with progression of CKD, with nearly twice in early CKD to 10-30 times of that of general population in patients with end-stage renal disease (ESRD). Additionally, conventional measures proven to be beneficial in the therapy of CVD, e.g., statins, anticoagulants, anemia, tight BP control, etc., have had little impact on mortality of CVD in CKD populations and in fact some of these therapies are associated with worsening outcomes.

Atherosclerosis is a state of generalized inflammation and any inflammatory state further worsens the process and progression of atherosclerosis. The current state of art in management of CVD and AS has focused primarily on treating the consequences of endothelial damage by attempting to reverse the pathology of AS. Thus, treatments affect metabolic processes that are secondary in nature, such as for example seeking to control lipids as a means to reduce the damage from LDL and related lipoproteins on preexisting lesions. Other areas of treatment focus include inhibiting platelet aggregation to reduce the chances of thrombosis and treatment of hypertension, for example. Other general conditions treatment is focused on include management of hypertension, anemia, dyslipidemia and diabetes but unfortunately, treatment of these conditions have had little impact on survival advantage in patients with CKD. These measures though important in therapy, do not address the primary environment of AS namely inflammation, insulin resistance, and endothelial dysfunction.

Though the CVD and CKD share many risk factors, existent therapeutic options including statins effective in management of CVD in the general population and have no effect in reducing mortality in patients with CKD, as efficient pathophysiologically-relevant treatment of CKD-related CVD is not available at present time.

Given the severity of CVD as a major component of patient care costs and a need to lower the incidence of CVD, there remains a long felt need for compositions and treatment methods to alleviate the effects, both monetary and clinical, of this and other disorders. As described below, the present invention provides novel compositions and treatment regimens for preventing and treating CVD and CKD-related conditions.

### DEFINITIONS

Before describing the instant invention in detail, several terms used in the context of the present invention will be defined. In addition to these terms, others are defined elsewhere in the specification as necessary. Unless otherwise expressly defined herein, terms of art used in this specification will have their art-recognized meanings.

Other features and advantages of the invention will be apparent from the following drawings, detailed description, and appended claims.

An “agent” refers to an active ingredient delivered to achieve an intended therapeutic benefit. As used herein, an agent can be any form of chloroquine compound, an example of which is hydroxychloroquine, or 4-aminoquinolines, that provide a benefit in the treatment of CVD.

The term “combination therapy” refers to a therapeutic regimen that involves the provision of at least two distinct therapies to achieve an indicated therapeutic effect. For example, a combination therapy may involve the administration of two or more chemically distinct active ingredients, or agents, for example, two forms of a chloroquine-based agent. Alternatively, a combination therapy may involve the administration of one or more chloroquine-based agents alone or in conjunction with another agent as well as the delivery of another therapy. In the context of the administration of two or more chemically distinct agents such as two more chloroquine agents, it is understood that the active ingredients may be administered as part of the same composition or as different compositions. When administered as separate compositions, the compositions comprising the different active ingredients may be administered at the same or different times, by the same or different routes, using the same or different dosing regimens, all as the particular context requires and as determined by the attending physician. Similarly, when one or more agents are combined with other drugs, the drug(s) may be delivered before, during, and/or after the period the subject is in therapy.

In the context of this invention, a “liquid composition” refers to one that, in its filled and finished form as provided from a manufacturer to an end user (e.g., a doctor, nurse, or patient), is a liquid or solution, as opposed to a solid. Here, “solid” refers to compositions that are not liquids or solutions. For example, such solids include dried compositions prepared by filtering, lyophilization, freeze-drying, precipitation, drying and similar procedures.

“Monotherapy” refers to a treatment regimen based on the delivery of one therapeutically effective composition, whether administered as a single dose or several doses over time.

A “patentable” composition, process, machine, or article of manufacture according to the invention means that the subject matter satisfies all statutory requirements for patentability at the time the analysis is performed. For example, with regard to novelty, non-obviousness, or the like, if later investigation reveals that one or more claims encompass one or more embodiments that would negate novelty, non-obviousness, etc., the claim(s), being limited by definition to “patentable” embodiments, specifically exclude the unpatentable embodiment(s). Also, the claims appended hereto are to be interpreted both to provide the broadest reasonable scope, as well as to preserve their validity. Furthermore, if one or more of the statutory requirements for patentability are amended or if the standards change for assessing whether a particular statutory requirement for patentability is satisfied from the time this application is filed or issues as a patent to a time the validity of one or more of the appended claims is questioned, the claims are to be interpreted in a way that (1) preserves their validity and (2) provides the broadest reasonable interpretation under the circumstances.

A “plurality” means more than one.

The term “species” when used in the context of describing a particular drug species, refers to a population of chemically indistinct molecules.

A “subject” or “patient” refers to an animal in need of treatment that can be effected by molecules of the invention. Animals that can be treated in accordance with the invention include vertebrates, with mammals such as bovine, canine, equine, feline, ovine, porcine, and primate (including humans and non-human primates) animals being particularly preferred examples.

The term “pharmaceutically acceptable salt” refers to salts which retain the biological effectiveness and properties of the compounds of the invention, and which are not biologically or otherwise undesirable. In many cases, the compounds of this invention are capable of forming acid and/or base salts by virtue of the presence of amino and/or carboxyl groups or groups similar thereto. Pharmaceutically acceptable acid addition salts may be prepared from inorganic and organic acids, while pharmaceutically acceptable base addition salts can be prepared from inorganic and organic bases. For a review of pharmaceutically acceptable salts, see, e.g., Berge, et al. (J. Pharm. Sci., vol. 66, 1 (1977)).

The expression “non-toxic pharmaceutically acceptable salts” non-toxic salts formed with nontoxic, pharmaceutically acceptable inorganic or organic acids or inorganic or organic bases. For example, the salts include those derived from inorganic acids such as hydrochloric, hydrobromic, sulfuric, sulfamic, phosphoric, nitric, and the like, as well as salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, fumaric, methanesulfonic, trifluoromethanesulfonic, and toluenesulfonic acid and the like. Salts also include those from inorganic bases, such as ammonia, sodium hydroxide, potassium hydroxide, and hydrazine. Suitable organic bases include methylamine, ethylamine, propylamine, dimethylamine, diethylamine, diethanolamine, trimethylamine, triethylamine, triethanolamine, ethylenediamine, hydroxyethylamine, morpholine, piperazine, and guanidine, as the case may be as use of such salts are amenable to compositions of chloroquine-based molecules.

The present invention also includes other forms of the compounds of the invention, including prodrug and polymorph forms. Here, a “prodrug” is a compound that contains one or more functional groups that can be removed or modified in vivo to result in a molecule that can exhibit therapeutic utility in vivo. A “polymorph” refers to a compound that has an identical chemical composition (i.e., it is of the same compound species) as compared to another compound but that differs in crystal structure. In preferred embodiments, the invention compounds comprising chloroquine-based molecules and other agents could be removed or modified in vivo or comprise the same composition as other compounds but differing in specific structure.

The term “effective amount” of a compound (or composition, or the like) means an amount that is effective to exhibit the desired biological activity or achieve the desired clinical result in a subject response to the particular treatment, commensurate with a reasonable benefit/risk ratio when used in the manner of this invention.

A “therapeutically effective amount” refers to an amount of an active ingredient sufficient to effect treatment when administered to a subject in need of such treatment. In the context of cancer treatment, a “therapeutically effective amount” is one that produces an objective response in evaluable patients. Such responses include changes in one or more parameters associated with CVD. Determination of therapeutically effective dosages of a composition comprising chloroquine-based molecules, according to the invention, may be readily made by those of ordinary skill in the art. Of course, the therapeutically effective amount will vary depending upon the particular subject and condition being treated, the weight and age of the subject, the severity of the disease condition, the particular compound chosen, the dosing regimen to be followed, timing of administration, the manner of administration and the like, all of which can readily be determined by one of ordinary skill in the art. It will be appreciated that in the context of combination therapy, what constitutes a therapeutically effective amount of a particular active ingredient may differ from what constitutes a therapeutically effective amount of the active ingredient when administered as a monotherapy.

The term “treatment” or “treating” means any treatment of a disease or disorder, including preventing or protecting against the disease or disorder (that is, causing the clinical symptoms not to develop); inhibiting the disease or disorder (i.e., arresting or suppressing the development of clinical symptoms; and/or relieving the disease or disorder (i.e., causing the regression of clinical symptoms). As will be appreciated, it is not always possible to distinguish between “preventing” and “suppressing” a disease or disorder since the ultimate inductive event or events may be unknown or latent. Accordingly, the term “prophylaxis” will be understood to constitute a type of “treatment” that encompasses both “preventing” and “suppressing”. The term “protection” thus includes “prophylaxis”.

### SUMMARY OF THE INVENTION

Turning now to the invention, in a first aspect, the invention comprises a treatment for prevention and amelioration of cardiovascular diseases in patients known or suspected to have chronic kidney disease. Specifically, the compositions and methods of the invention are effective in ameliorating the initiation and progression of AS in patients having or at risk of having CKD. In preferred embodiments, the amelioration comprises reducing systemic inflammation, improving or restoring vascular endothelial function, and/or insulin resistance in such subjects.

In a second aspect, the present invention provides compositions and methods that comprise treating multiple arms of AS such as, for example, inflammation, insulin resistance, endothelial reactivity, and health.

In a third aspect, the present invention provides benefit to patients exhibiting CVD or that have a predisposition to CVD as well as patients with proven CVD and CKD who have a many fold increase in CVD related morbidity and mortality.

In still other aspects, the invention contemplates use of formulations that include chloroquine-based compounds, such as 4-aminoquinoline, chloroquine, and hydroxychloroquine (HCQ), routes, dosages, and treatment regimens delivering such formulations for various ailments either directly or indirectly related to CVD.

### DETAILED DESCRIPTION OF THE PREFERRED EMBODIMENTS

As those in the art will appreciate, the following description describes certain preferred embodiments of the invention in detail, and is thus only representative and does not depict the actual scope of the invention. Before describing the present invention in detail, it is understood that the invention is not limited to the particular device arrangements, systems, and methodologies described, as these may vary. It is also to be understood that the terminology used herein is for the purpose of describing particular embodiments only, and is not intended to limit the scope of the invention defined by the appended claims.

In a first aspect, the invention concerns a treatment for prevention, repair and amelioration of cardiovascular diseases. Specifically, the compositions and methods of the invention are effective in ameliorating the initiation and progression of AS. In a preferred embodiment the amelioration comprises reducing systemic inflammation, improving or restoring vascular endothelial function, and insulin resistance by administering to a patient in need thereof of an effective amount of a composition comprising a chloroquine-based molecule.

Chloroquine-based compounds, such as hydroxychloroquine (HCQ), have antimalarial and anti-inflammatory properties. HCQ is routinely used as a standard of care in many active rheumatological disorders for control of systemic inflammation, disease activity and relapses. In vivo and in vitro studies have examined the pharmacological properties of HCQ that have relevance in cardiovascular physiology.

Over the past 20 years several studies worldwide and testing multiple cohort-based trials have repeatedly shown that HCQ use in various inflammatory diseases provides significant survival advantages. For example, a test from a Spanish trial cohort, HR, for death from any cause was 0.14. In this study 232 patients demonstrated zero cardiovascular deaths occurred in patients using HCQ over the 52-month study period whereas 7 deaths were noted in those never exposed to HCQ. In another cohort (the Lumina cohort) from southern US, the OR for death was 0.31. In a Latin American cohort, OR, for death was 0.62. In related studies, such as by Shinjo et al., the Gladel cohort suggests that HCQ provides a time-dependent beneficial effect with progressive reduction in mortality risk with increasing duration of HCQ use. Moreover, HCQ use was associated with improvements in parameters relevant to CVD namely, decrease in vascular stiffness, reduction in the incidence of, or the new onset of hypertension, metabolic syndrome (OR, 0.13), and vascular events (OR, 0.3) and overall reduction in CVD.

These studies in addition to various retrospective analyses of rheumatological disease databases suggest that HCQ used as an anti-inflammatory agent may provide for reduction in mortality rates and may further have potential cardiovascular advantages as well. Additionally, it has recently been demonstrated in an APO E −/− model that Chloroquine, a related molecule to HCQ, has significant atheroprotection activity through the p53 dependant mechanisms showing the reduction in atherosclerotic burden in mice treated with chloroquine. To date, HCQ and other chloroquine compounds have not been applied to therapies for AS and CVD in patients exhibiting CKD. Indeed, in the context of this invention certain chloroquine compounds have been discovered to be active in ameliorating CVD in CKD patients, such as for example 4-aminoquinoline compounds.

In second embodiment, the present invention compositions and methods comprise treating multiple arms of AS such as for example, inflammation, insulin resistance, endothelial reactivity and health in patients predisposed to or expressing naive or chronic kidney disease.

In another aspect, the present methods provide benefit to patients exhibiting CVD with naïve CKD or that have a predisposition to CVD as well as patients with proven CVD and CKD who have a many fold increased occurrence of CVD related morbidity and mortality.

In still aspects, the invention contemplates use of formulations, routes of administration, dosages, and treatment regimens for various ailments either directly or indirectly related to CVD.

Thus, a further aspect of this invention concerns treatment compositions, particularly pharmaceutical or veterinary compositions, comprising a chloroquine-based molecule or an acceptable salt, base, or prodrug form thereof, formulated together with one or more non-toxic acceptable carriers, preferably pharmaceutically acceptable carriers. The terms “pharmaceutically acceptable carrier” and “physiologically acceptable carrier” refer to molecular entities and compositions that are physiologically tolerable and do not typically produce an unintended allergic or similar untoward reaction, such as gastric upset, dizziness and the like, when administered to a subject. In the context of therapeutic compositions intended for human administration, pharmaceutically acceptable carriers are used. The compounds of the invention may be processed in accordance with conventional methods of pharmaceutical compounding techniques to produce medicinal agents (i.e., medicaments or therapeutic compositions) for administration to subjects, including humans and other mammals, i.e., “pharmaceutical” and “veterinary” administration, respectively. See, for example, the latest edition of Remington's Pharmaceutical Sciences (Mack Publishing Co., Easton, Pa.). Typically, a composition such as one comprising chloroquine type molecules is combined as a composition with a pharmaceutically acceptable carrier. The composition(s) may also include one or more of the following: excipients; preserving agents; solubilizing agents; stabilizing agents; wetting agents; emulsifiers; sweeteners; colorants; odorants; salts; buffers; coating agents; and antioxidants.

Further still, the compounds, and their respective acid or base salts, can be formulated into liquid, preferably aqueous, formulations for storage and administration, as well as dried formulations that may, for example, be used as powders for intranasal administration or be reconstituted into liquid form just prior to administration to a subject. Liquid pharmaceutically administrable compositions can, for example, be prepared by dissolving, dispersing, etc. the particular active compound and optional pharmaceutical adjuvants in an aqueous carrier. Aqueous carriers include water (particularly water for injection into humans), alcoholic/aqueous solutions, and emulsions and suspensions. Preferred pharmaceutically acceptable aqueous carriers include sterile buffered isotonic saline solutions. Parenteral vehicles include sodium chloride solution, Ringer's dextrose, dextrose, and sodium chloride, lactated Ringer's, or fixed oils. Intravenous vehicles include fluid and nutrient replenishers, electrolyte replenishers (such as those based on Ringer's dextrose), and the like. Preservatives and other additives may also be present, such as, for example, antimicrobials, antioxidants, chelating agents, and inert gases and the like. Non-aqueous solvents may also be included, although when included they preferably comprise less than about 50%, more preferably less than about 25%, and even more preferably less about 10%, of the total solvent of the solution. Examples of non-aqueous solvents include propylene glycol, ethanol, polyethylene glycol, vegetable oils such as olive oil, and injectable organic esters such as ethyl oleate. The pharmaceutical and veterinary compositions of the invention, whether dry or liquid, are preferably formulated for oral administration.

If desired, a composition useful in the context of the invention to be administered may also contain minor amounts of nontoxic auxiliary substances such as wetting agents, emulsifying agents, or solubilizing agents, antioxidants, antimicrobials, pH buffering agents and the like, for example, sodium acetate, sodium citrate, cyclodextrin derivatives, sorbitan monolaurate, triethanolamine acetate, triethanolamine oleate, etc. Actual methods of preparing such dosage forms are known, or will be apparent, to those skilled in this art; for example, see Remington: The Science and Practice of Pharmacy, Mack Publishing Company, Easton, Pa., 20th Edition, 2000. The composition or formulation to be administered will, in any event, contain a quantity of the active compound in an amount effective to alleviate the symptoms of the subject being treated.

As those in the art will appreciate, the compounds of the invention may also be formulated for targeted delivery of the active ingredient to a subset of tissues or cells in a subject. In general, targeted delivery is accomplished by formulating a compound of the invention with a targeting moiety. Such moieties include lipids, liposomes, and ligands for molecules that bind, or are bound by, other molecules in vivo.

A composition is comprised of “substantially all” of a particular compound, or a particular form a compound (e.g., an isomer) when a composition comprises at least about 90%, and preferably at least about 95%, 99%, and 99.9%, of the particular composition on a weight basis. A composition comprises a “mixture” of compounds, or forms of the same compound, when each compound (e.g., isomer) represents at least about 10% of the composition on a weight basis. A treatment composition comprising a form of chloroquine, or a conjugate thereof, can be prepared as an acid salt or as a base salt, as well as in free acid or free base forms. In solution, certain of the compounds of the invention may exist as zwitterions, wherein counter ions are provided by the solvent molecules themselves, or from other ions dissolved or suspended in the solvent.

The amount of the compound required for use in treatment will vary not only with the particular extract composition and salt selected, but also with the route of administration, the nature of the condition being treated, and the age and condition of the patient, among other factors, and ultimately is determined at the discretion of the attending physician or clinician. The desired dose may conveniently be presented in a single dose or as divided doses administered at appropriate intervals, for example, as two, three, four or more sub-doses per day. The sub-dose itself may be further divided, for example, into a number of discrete, loosely spaced administrations, such as by ingesting multiple pill or liquid doses.

### Administration

The compounds of this invention are administered in a therapeutically effective amount to a subject in need of treatment. Administration of the compositions of the invention can be via any of suitable route of administration, particularly by ingestion, or alternatively parenterally, for example, intravenously, intra-arterially, intraperitoneally, intrathecally, intraventricularly, intraurethrally, intrasternally, intracranially, intramuscularly, intranasally, rectally, subcutaneously, sublingually, transdermally, or by inhalation or insufflation. Such administration may be as a single oral dose or a bolus injection, multiple injections, or as a short- or long-duration infusion. Implantable devices (e.g., implantable infusion pumps) may also be employed for the periodic parenteral delivery over time of equivalent or varying dosages of the particular formulation. For such parenteral administration, the compounds are preferably formulated as a sterile solution in water or another suitable solvent or mixture of solvents. The solution may contain other substances such as salts, sugars (particularly glucose or mannitol), to make the solution isotonic with blood, buffering agents such as acetic, citric, and/or phosphoric acids and their sodium salts, and preservatives. The preparation of suitable, and preferably sterile, parenteral formulations is described in detail in the section entitled “Compositions”, above.

In the context of this invention, actual dosage levels for the compositions of this invention can be varied so as to obtain an amount of the active compound(s) that is effective to achieve the desired therapeutic response for a particular patient, compositions and mode of administration. In general, daily administration or continuous infusion at dosages less than those known to produce toxicities will be the preferred therapeutic protocol to enhance the activity of the drug. The selected dosage level will depend upon the activity of the particular compound, the route of administration, the severity of the condition being treated and the condition and prior medical history of the patient being treated. However, it is within the skill of the art to start doses of the compound at levels lower than required to achieve the desired therapeutic effect and to gradually increase the dosage until the desired effect is achieved.

With regard to human and veterinary treatment, the amount of a particular composition that is administered will, of course, be dependent on a variety of factors, including the disorder being treated and the severity of the disorder; activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; the judgment of the prescribing physician or veterinarian; and like factors well known in the medical and veterinary arts.

In further embodiments, the extract comprises treatment formulations that can be made in powdered form for administration via ingestion, with or without additional ingredients, such as dietary supplements, comprising combination formulas with vitamins, minerals and other nutritional supplements.

Dosage forms include tablets, troches, dispersions, suspensions, solutions, capsules, patches, and the like. The compound may be administered prior to, concurrently with, or after administration of other therapies, or continuously, i.e., in daily doses, during all or part of, a separate therapy regimen from that of treating CVD or AS or treating for prevention of CVD or AS. The compound, in some cases, may be combined with the same carrier or vehicle used to deliver the anti-hearing loss therapeutic agent.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavouring agent such as peppermint, oil of wintergreen, or cherry flavouring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. Tablets, capsules, pills, granules, microparticles and the like can also comprise an enteric coating, such as a coating of one of the Eudragit® polymers, that will permit release of the active compound(s) in the intestines, not in the acidic environment of the stomach.

A syrup or elixir may contain the active compound, sucrose or fructose as a sweetening agent, methyl and propylparabens as preservatives, a dye and flavouring such as cherry or orange flavor. Of course, any material used in preparing any unit dosage form should be pharmaceutically acceptable and substantially non-toxic in the amounts employed. In addition, the active compound or composition of compounds, such as a formula of chloroquine and hydroxychloroquine, for example, may be incorporated into sustained-release preparations and devices.

The active compound or composition may also be administered intravenously or intraperitoneally by infusion or injection. Solutions of the active compound or its salts can be prepared in water, optionally mixed with a non-toxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form must be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, non-toxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions can be prepared by incorporating the active compound(s) in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the compounds may be applied in liquid or cream-based formulations, which preferably will include a dermatologically acceptable carrier, which may be a solid, gel, or liquid. Useful solid carriers include finely divided solids such as talc, clay, microcrystalline cellulose, silica, alumina and the like. Useful liquid carriers include water, alcohols, or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers. Thickeners such as synthetic polymers, fatty acids, fatty acid salts and esters, fatty alcohols, modified celluloses, and/or modified mineral materials can also be employed with liquid carriers to form spreadable pastes, gels, ointments, soaps, and the like, for application directly to the skin of the user.

Useful dosages of the compounds of the invention can be determined by comparing their in vitro activity, and in vivo activity in animal models. Methods for the extrapolation of effective dosages in mice, and other animals, to humans are known to the art. See, e.g., U.S. Pat. No. 4,938,949.

Other drugs or treatments, including treatment with other agents such as chemotherapeutic agents, irradiation, or other anti-cancer agents such as alkylating agents, anti-tumor antibodies, or cytokines, can be used with the present compounds. See, e.g., Remington's Pharmaceutical Sciences (18^{th} ed. 1990) at pages 1138-1162.

In further embodiments, the treatment of CVD in CKD challenged patients provides for a potential benefit in numerous aspects of physiology that result in lowered mortality. For example, the ability for chloroquine compounds to aid CVD conditions is well documented. For example, the primary anti-inflammatory action of chloroquine, such as hydroxychloroquine, seems to stem from its lysosomotropic actions with resultant reduction in local inflammation. The role of lysosomes in the handling and degradation of modified LDLs and oxidative stress is established and can be potentially benefited by hydroxychloroquine through its stabilization of lysosomes. Additionally, in another example, matrix metallo proteinase-9 (MMP-9) which is an established marker of the state of inflammation associated with AS, and an independent marker of CVD, was inhibited in its secretion in Systemic lupus erythematosus (SLE) patients by administration of hydroxychloroquine. One proposed mechanism of action is the possible role of hydroxychloroquine in inhibiting mRNA synthesis with resultant reduction in inflammation.

Further potential benefits of chloroquine-based compound administration include potential improvements in insulin sensitivity and metabolic syndrome wherein hydroxychloroquine can improve insulin sensitivity and benefit metabolic syndrome, as shown in experimental models. Here, the hydroxychloroquine lowered fasting glucose in patients with inflammatory disorders on a longitudinal cohort analysis.

Additional potential benefits of hydroxychloroquine administration include improvement in endothelial function. For example, defects in nitric oxide (NO) availability or physiological action with resultant endothelial dysfunction is found in early vascular injury of AS. Such dysfunction is found much earlier than onset of structural and clinical AS syndromes. Treatment with hydroxychloroquine provides for better indices of vascular function such as for example, improved NO release and action and reduction in vascular stiffness. For example, in various lupus cohorts improvement was observed in vascular stiffness and in endothelial NO synthesis and release as well as endothelial mediated vascular reactivity. Further, in another example, in a study of lupus in premenopausal women, hydroxychloroquine use was associated with better aortic pulse wave velocity (APWV), now an established marker for CVD and mortality in both the general population and CKD patient populations.

Still further potential benefits include reduction in new onset of hypertension, test subjects exhibiting a lowered blood pressure after regimens of hydroxychloroquine. Also, improvement in dyslipidemia has been observed in rheumatological disorders showing reduction in total cholesterol, LDL, IDL, and improvement in HDL.

Further potential benefits for artheroprotective action of chloroquine compounds are evidenced in animal studies. For example, Ataxia Telengectesia Mutated (ATM)-deficient mice, which exhibit glucose intolerance, elevated blood pressure, atherosclerosis, and insulin resistance, and various components of the metabolic syndrome, showed when treated with chronic low dose administration of, activates ATM/p53 axis in many tissues, including adipose, liver, and the vasculature through the Jun N-terminal kinase (JNK), a major mediator of inflammation, insulin resistance, and vascular disease and has beyond this potent atheroprotective activity.

In a particularly preferred embodiment, the dosages of chloroquine containing compounds, such as for example, hydroxychloroquine, for treating CVD and AS can comprise chronic low dosage regimens of between 100 and 400 mg, more usually between 100 and 300 mg and even more usually about 200 mg per dose administered twice per day for adults with average 1.73 m2 BSA.

### Applications

As described above, certain aspects of the invention relate to compositions that contain a compound of the invention, which compositions are useful in the treatment or prevention of CVD in, for example, humans or other mammals (e.g., bovine, canine, equine, feline, ovine, and porcine animals), and other animals as well, particularly those having or at risk of having or suspected to have CKD. Specifically, this invention enables the treatment of patients predisposed to or exhibiting kidney disease, particularly chronic kidney disease for ameliorating CVD. In the context of such therapy, the compounds of the present invention may be used alone, i.e., in monotherapy, or in combination with other therapeutic agents such as, for example, anti-cancer therapies (e.g., radiation, surgery, bone marrow transplantation, etc.), that involve use of drugs that are potentially detrimental to the cardiovasculature. As will be appreciated, “combination therapy” and the like refer to a course of therapy that involves the provision of at least two distinct therapies to achieve an indicated therapeutic effect. For example, a combination therapy may involve the administration of two or more chemically distinct active ingredients, for example, a fast-acting chemotherapeutic agent and a myeloprotective agent. The agents may be delivered or may be administered as part of the same composition or as different compositions according to the same therapeutic regimen or different regimens, depending on the active ingredients involved, the disease to be treated, the age and condition of the patient, etc. Moreover, when used in combination with another therapeutic agent, the administration of the two agents may be simultaneous or sequential. Simultaneous administration includes the administration of a single dosage form that comprises both agents, and the administration of the two agents in separate dosage forms at substantially the same time. Sequential administration includes the prior, concurrent, or subsequent administration of the two or more agents according to the same or different schedules, provided that there is an overlap in the periods during which the treatment is provided.

### Example 1

### Treatment for Cardiovascular Disease

### Introduction:

Cardiovascular disease (CVD) is the largest killer of people in U.S., and is only partially explained by conventional CV risk factors. Chronic kidney disease (CKD) is a major non-traditional CV risk factor and is associated with progressive and exponential increase in CV morbidity and mortality. The presence of multiple traditional and nontraditional risk factors, including inflammation, contribute to accelerated atherosclerosis (AS) in patients with CKD.

Hydroxychloroquine (HCQ) is administered as an anti-inflammatory in rheumatological disorders, and it has multiple beneficial properties relevant to the process of atherogenesis, as established in vivo and in vitro experiments. These include improvements in insulin sensitivity, vascular compliance, lipid disorders, metabolic syndrome, and inflammation. Recently HCQ has been demonstrated to have significant atheroprotection in APO E −/− mice, and analysis of multiple rheumatological cohorts with CVD proves major mortality and CVD benefits (OR for death 0.12-0.49).

Thus, the purpose of this study will be to prospectively evaluate for the first time HCQ in treating CVD, including examining the effects of HCQ on biological and functional end points relevant to atherosclerosis and CVD in patients with CKD who represent a high-risk population. This is a randomized double blind placebo controlled trial with 3:1 (HCQ:placebo) allocation in patients with advanced (Stage IV) CKD. The data obtained in this study would provide rationale and support for planning studies to examine the effect of HCQ on clinically relevant hard end points of CV disease in CKD population.

This study will be a randomized double blind placebo controlled trial with 3:1 (HCQ: placebo) allocation in 52 patients (39 HCQ group and 13 placebo group) with advanced (Stage IV) CKD with the following end points: aortic stiffness (aortic pulse wave velocity; APWV); endothelial injury and/or dysfunction (sVCAM-1, endothelial progenitor cells, and endothelial microparticles), inflammatory measures (IL-6 and hs-CRP), oxidative stress (oxLDL), other relevant markers (e.g., Cardiac troponin-T and cTNT), and progression of CKD (eGFR decline). The levels of HCQ and metabolites will also be monitored at regular intervals (at 1, 3 and 6 months), and the overall safety of HCQ administration in this population will also be assessed. Finally, various relevant cardiac and renal end points will be monitored, including: levels of asymmetrical dimethylarginine, HDL, LDL, N-Terminal proBNP; systolic blood pressure; primary cardiovascular events; initiation of dialysis; and all-cause mortality.

### Methods:

Study Population:

A prospective, double blind, randomized, controlled trial will be undertaken with 3:1 (HCQ:placebo) allocation with a total of 52 subjects, 18 to 80 years, with stage IV proteinuric CKD with an estimated MDRD GFR (eGFR) of to 20 to 30 ml/min, who have either history of, or current documented proteinuria of more than 1 gm in 24 hours or a spot urine protein to creatinine ratio of greater than 1. This population with advanced CKD and significant proteinuria has a high CV burden, more than 4-fold higher as compared to general population. Individuals with active or recent infection, active systemic inflammatory diseases, e.g., SLE, vasculitis, prior transplantation, pregnancy or breast feeding, active or recently treated malignancy, known prolongation of QT interval, G6PD deficiency, pre-existing significant liver disease or cirrhosis, recent unexplained changes in vision and documented poor compliance towards either clinic attendance or medication, will be excluded. Demographic, clinical, and laboratory parameters will be obtained at the baseline prior to initiation of treatment, after one month, 3 months, and 6 months of treatment.

Choice of Intervention:

Chloroquine (CHQ) is an antimalarial drug with anti-inflammatory properties and has had a good safety profile established over the last 4 decades. Hydroxychloroquine (HCQ) prepared by β hydroxylation of CHQ. HCQ has better safety profile and is used to treat many rheumatological disorders. It is available as 200 mg tablet (155 mg of base); is absorbed rapidly and completely, has moderate plasma protein binding, and is metabolized by the liver with an elimination half-life of 32-50 days. Long term, HCQ is a well tolerated drug, except for some minor side effects, including cutaneous or GI side effects and, in humans, significant HCQ toxicity is uncommon to rare. It has been used frequently even in ESRD for variety of diseases, e.g. RA, SLE, and nephrogenic systemic fibrosis. Although no drug dose adjustment has been recommended for patients with CKD having eGFR greater than 10, the dose used will be that has been shown to be efficacious in most patients but is less than the maximum recommended dose. In addition, the blood levels of CHQ and metabolites will be monitored.

Statistical Methods:

To determine whether the treatment moves biomarkers in an ameliorative direction to a greater degree than placebo, each biomarker will be analyzed. Beyond considering each biomarker alone, they will also be considered jointly in the following three steps. (i) for each of the 9 primary biomarkers, pre- to post-trial change experienced by the individuals will be examined; (ii) these changes will be standardized within a biomarker so that the mean change among all subjects is 0 and the SD is 1. These standardized values will be “z-scores”; and (iii) the vector of these changes will be analyzed in a two-factor ANOVA accounting for treatment, type of biomarker, and their interaction. The primary test is the treatment effect. Since a vector of 9 z-scores comes from a single individual, repeated measures methodology will be used. As a secondary analysis, the changes may be classified into beneficial, detrimental, or no change, based on clinical criteria specific to the measure. These vector categorical changes will be compared between the two arms within a generalized linear mixed model that accounts for the within-individual correlation.

Process of Randomization and Blinding:

The process of randomization and blinding will be achieved through the packaging of commercially available HCQ tablets in empty capsules with matching placebo packaging in similar capsules. Patients randomized to HCQ will be started on capsule containing the dose of 200 mg by mouth, once a day for an initial 10 days followed by increase to twice a day for a period of 24 weeks, while those randomized to usual care will receive a placebo in similar fashion. The blinding results will not be shared with physicians, patients, or epidemiologists.

Choice of Primary End Points:

Extensive review of the literature was performed to evaluate the variety of markers available, and each individual marker was analyzed with respect to its strength of association with CVD beyond that provided by conventional parameters, including its distribution variation, superiority over contemporaneous measures interaction with co-morbidities, stability and reproducibility in measurement, and utility to reflect the dynamic state of AS and CVD over time (both in general population and in patients with CKD). The combination of markers was selected to ensure a capacity to reflect different major pathogenetic pathways for AS and CVD, as described below.

Based on the detailed criteria described above the following primary end points were chosen to reflect the effect of HCQ on the different processes relevant to AS and CVD.
A. Vascular Functional Marker; APWV: it has a strong correlation with CVD and mortality, both in general population and patients with CKD and ESRD, and is now recommended over other modalities evaluating the vascular stiffness. Additionally, it predictably improves with measures of proven CV benefit both in general population and in CKD over 6-12 months of therapy.
B. Endothelial Function; Biochemical Markers: ADMA (a naturally occurring amino acid with ability to inhibit all three forms of NO Synthetase) and sVCAM-1 (a principal effector of monocyte migration) have been shown to be elevated in patients with CKD, have strong and independent effects on CV events and death both in general population and CKD and favorably improves with therapies of proven CV benefits (statins, ACEi and ARB) independent of their BP lowering effects and with renal transplantation).
C. Cellular Markers of ongoing Vascular Injury and Repair: Vascular injury (reflected by increased circulating EMPs, CD 144+/CD146+) or defective repair (reflected by reduced Bone Marrow derived CD34+ precursor cells, Endothelial progenitor cells with co-localizing Endothelial marker e.g. KDR/Flt-1, VEGF-2 etc) are excellent marker of dynamic state of endothelial health and currently explored for their therapeutic potential. Reduction in EPC, increase in EMPs, or an imbalance between these two has been strongly shown to be associated with adverse cardiac events and are favorably impacted interventions with proven CV benefits e.g. statins, RAAS blockers and in ESRD arena with nocturnal dialysis.
D. Inflammatory Markers: IL-6 and hs-CRP have both association and putative causality for the process of AS, with IL-6 having a stronger predictive value than CRP for cardiovascular and all cause mortality in patients with CKD. Both IL-6 and CRP have been used as a tool for titration of CV therapies.
E. Modified LDLs: Modified LDLs and oxidative stress have a long history in the pathogenesis of AS.
F. Conventional CV markers: cTNT and NTproBNP are traditionally used markers for CV end points.
G. Renal specific Markers: the comparative decline in MDRD eGFR and cystatin C will be monitored for the duration of the study.
   To monitor toxicity, G6PD levels will be monitored in all patients.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the spirit and scope of the invention. More specifically, the described embodiments are to be considered in all respects only as illustrative and not restrictive. All similar substitutes and modifications apparent to those skilled in the art are deemed to be within the spirit and scope of the invention as defined by the appended claims.

All patents, patent applications, and publications mentioned in the specification are indicative of the levels of those of ordinary skill in the art to which the invention pertains. All patents, patent applications, and publications, including those to which priority or another benefit is claimed, are herein incorporated by reference to the same extent as if each individual publication was specifically and individually indicated to be incorporated by reference.

The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that use of such terms and expressions imply excluding any equivalents of the features shown and described in whole or in part thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A method selected from the group consisting of:
(a) a method of preventing cardiovascular disease (CVD) in a patient known to have or suspected of having chronic kidney disease (CKD);
(b) a method for reducing cardiovascular disease severity in a patient known to have or suspected of having CKD; and
(c) a method of preventing cardiovascular disease progression in a patient known to have or suspected of having chronic kidney disease (CKD),
comprising administering to a patient known to have or suspected of having CKD an effective amount of chloroquine or a chloroquine derivative compound to prevent, reduce the severity of, or prevent the progression of CVD.

2. A method according to claim 1 wherein the chloroquine derivative compound is selected from the group consisting of hydroxychloroquine and 4-aminoquinoline.

3. A method according to claim 1 wherein the chloroquine derivative compound is hydroxychloroquine.

4. A method according to claim 3 wherein the effective amount of hydroxychloroquine comprises about 100 to about 400 mg of hydroxychloroquine per day.

5. A method according to claim 4 wherein hydroxychloroquine is administered more than once per day.

6. A method according to claim 4 wherein about 200 mg of hydroxychloroquine is administered twice per day.

7. A method according to claim 1 wherein the cardiovascular disease is selected from the group consisting of myocardial infarction, congestive heart failure, and stroke.

8. A method according to claim 1 wherein preventing cardiovascular disease progression comprises reducing need for a revascularization procedure.

9. A method according to claim 1 that comprises administration of chloroquine or a chloroquine derivative compound to the patient for at least about 1-4 weeks, about 1-12 months, and about 1-10 or more years.

10. A method of cardioprotection in a patient known to have or suspected of having chronic kidney disease (CKD), comprising administering to a patient known to have or suspected of having CKD a cardioprotective amount of chloroquine or a chloroquine derivative compound.

11. A method of reducing risk of mortality in a patient known to have or suspected of having chronic kidney disease (CKD), comprising administering to a patient known to have or suspected of having CKD an amount of chloroquine or a chloroquine derivative compound effective to reduce risk of patient mortality, optionally patient mortality associated with cardiovascular disease (CVD).
